(19) United States
(12) Patent Application Publication
(10) Pub. No.: US 2026/0295137 A1
(43) Pub. Date: Oct. 1, 2026

(54) METHOD FOR REMOVING UREMIC TOXINS FROM BLOOD

(71) Applicants: INSTITUT NATIONAL DE LA SANTÉ ET DE LA RECHERCHE MÉDICALE, PARIS (FR); UNIVERSITE CLAUDE BERNARD LYON 1, VILLEURBANNE (FR); INSTITUT NATIONAL DE RECHERCHE POUR L'AGRICULTURE, L'ALIMENTATION ET L'ENVIRONNEMENT, PARIS (FR); HOSPICES CIVILS DE LYON, LYON (FR)

(72) Inventors: Christophe SOULAGE, BRON (FR); Fitsum GUEBRE-EGZIABER, BRON (FR); Nans FLORENS, STRASBOURG (FR)

(21) Appl. No.: 19/477,229

(22) PCT Filed: May 2, 2024
(86) PCT No.: PCT/EP2024/062166

§ 371 Date: Oct. 21, 2025

(30) Foreign Application Priority Data
May 3, 2023 (WO) PCT/EP2023/061643

(51) Int. Cl. A61M 1/36

(52) U.S. Cl. CPC A61M 1/3687; A61M 1/3661; A61M 2202/0498

(57) ABSTRACT

The invention relates to a method for removing uremic toxins bound to a protein from the blood of a patient in need thereof. The method comprises the introduction in the blood of a displacer substance chosen from (C6-C12)-fatty acids, salts of (C6-C12)-fatty acids, a precursor of said (C6-C12)-fatty acids, or mixtures thereof. The displacer substance replaces the uremic toxins bound to the protein, thereby releasing unbound uremic toxins in the blood. The unbound uremic toxins are then removed from the blood by any method of dialysis. The method according to the invention involves low concentrations of displacer substance and is effective at plasma concentrations comprised in the range of from 1 μM to 3 mM.

### TECHNICAL FIELD

The present invention relates to a method for removing uremic toxins from blood, and in particular relates to a dialysis method allowing the removal of protein bound uremic toxins from the blood of a patient in need of dialysis.

### BACKGROUND OF THE INVENTION

The kidney has different functions such as the removal of waste products from biological fluids such as blood. Failure of the kidney associated with the accumulation of a large range of uremic toxins, leading to many deleterious biological effects. Removal of these toxins is important and is often carried out by dialysis methods such as hemodialysis, peritoneal dialysis, hemofiltration or hemodiafiltration.

Uremic toxins constitute a group of substances which are retained in patients with kidney failure and are involved in the development of uremic syndrome. Accumulation of uremic toxins interfere with the normal functioning of many organs and exhibits an important toxicity regarding the cardiovascular system, thereby contributing to increased cardiovascular risk in patients with kidney failure.

Uremic toxins can be divided in three major groups: first, the free water-soluble low molecular weight molecules (MW<500 Da, prototype of which being urea), easily removed by standard renal replacement therapies. Second, middle molecules (0.5-60 kDa, prototype of which being Beta-2 microglobulin) for which removal was improved with high cut off dialysis membranes and high-volume convection therapies. In recent years, focus has however shifted toward the third category, consisting in protein-bound uremic toxins (PBUT). These latter are characterized by their extensive toxicity, while their removal strategies are still limited by their tight binding to serum albumin.

Indoxyl sulfate (IS) and p-Cresyl sulfate (p-CS) represent prototypes of PBUTs and as such have been extensively studied. They were associated with all-cause mortality, cardiovascular disease, as well as worsening of renal function in chronic kidney disease (CKD) patients.

Thus, their removal is a major concern in patients with end stage renal disease (ESRD). The difficulty is that only the free fraction of PBUTs (namely less than 10%) is available for diffusion across the dialysis membrane. Their binding to albumin involves non-covalent reversible bindings such as electrostatic or hydrophobic interactions, Van de Waals forces or hydrogen bounds. The increase of the free fraction of PBUT by using chemicals, i.e. displacer substances, that compete with PBUT for binding onto albumin sites could therefore be a valuable option to increase the free fraction and thus improve their clearance in dialysis patients.

Several of such displacer substances have been identified, but their use has often been found to be problematic.

For instance, unsaturated long chain fatty acids, such as oleic acid and linoleic acid have been considered, but their lipophilic feature makes them impractical for use in hemodialysis, and because they are known to exacerbate dyslipidemia.

Fatty acids of shorter chains have also been proposed. Octanoate has been shown to displace affinity for the Sudlow II site of albumin, but at high concentrations, such as concentrations of 0.24 M, rendering their use incompatible with an in vivo dialysis procedure.

Other substances, such as for example ibuprofen or furosemide, have also been considered, but many of these substances show either ineffective displacing properties, needing high concentrations to be effective, and/or are toxic for the patient, causing for instance hemolysis at relatively low concentrations, rendering their use in hemodialysis inadequate.

A need therefore exists for the development of displacer substances compatible with a dialysis procedure, showing effective displacement properties of uremic toxins at low concentrations, whilst being non-toxic to the patient and easily administered.

### SUMMARY OF THE INVENTION

The present invention solves the above-mentioned problems. The inventors have identified, after extensive research, displacer substances that effectively displace protein bound uremic toxins at low plasma concentrations, i.e. 1 μM-3 mM, such as in the range of 0.5-3 mM, whilst at the same time being compatible with the patient's blood, thus allowing their safe use in a dialysis process.

It is thus proposed a method for removing uremic toxins bound to a protein from the blood of a patient in need thereof, the method comprising the steps of:
a) Introducing a displacer substance in the blood, under conditions in which the displacer substance replaces the uremic toxins bound to the protein, thereby releasing unbound uremic toxins in the blood,
b) Removing the unbound uremic toxins from the blood by dialysis,
   the displacer substance being chosen from (C6-C12)-fatty acids, salts of (C6-C12)-fatty acids, a precursor of said (C6-C12)-fatty acids, or mixtures thereof,
   the displacer substance being provided in the blood at a plasma concentration comprised in the range of from 1 μM to 3 mM, in particular of from 0.5 to 3 mM.

### BRIEF DESCRIPTION OF THE FIGURES

Other features, details and advantages will be shown in the following detailed description and on the figures, on which:

FIG. 1 shows an experimental setup for in vitro hemodialysis assay according to example 5.

FIG. 2 shows an in vivo setup 10 able to be used to implement a hemodialysis method according to the invention. The setup 10 comprises a dialyzer 11, an arterial line 12, a venous line 13, a blood flow controller 14, a dialysate flow controller 15 and a syringe 16 comprising a solution or emulsion of a displacer substance.

FIG. 3 shows the results of the ultrafiltration assay according to example 2. FIG. 3A shows the percentage of free uremic toxin IS as a function of the concentration, expressed in mM, of the displacer substance sodium hexanoate. FIG. 3B shows the percentage of free uremic toxin IS as a function of the concentration, expressed in mM, of the displacer substance sodium octanoate. FIG. 3C shows the percentage of free uremic toxin IS as a function of the concentration, expressed in mM, of the displacer substance sodium decanoate. FIG. 3D shows the percentage of free uremic toxin IS as a function of the concentration, expressed in mM, of the displacer substance sodium decanoate. Asterix indicate a significant difference from control condition: *P<0.05, ** P<0.01, *** P<0.005.

FIG. 4 shows the results of the ultrafiltration assay according to example 2. FIG. 4A shows the percentage of free uremic toxin p-CS as a function of the concentration, expressed in mM, of the displacer substance sodium hexanoate. FIG. 4B shows the percentage of free uremic toxin p-CS as a function of the concentration, expressed in mM, of the displacer substance sodium octanoate. FIG. 4C shows the percentage of free uremic toxin p-CS as a function of the concentration, expressed in mM, of the displacer substance sodium decanoate. Asterix indicate a significant difference from control condition: *P<0.05, ** P<0.01, *** P<0.005.

FIG. 5 shows the result of a competition test between two medium chain fatty acids (octanoate and decanoate) and p-Cresyl-sulfate (p-CS) for binding to human serum albumin, according to example 3. FIG. 5A shows the percentage of free uremic toxin p-CS as a function of the concentration, expressed in mM, of the displacer substance octanoate. FIG. 5B shows the percentage of free uremic toxin p-CS as a function of the concentration, expressed in mM, of the displacer substance decanoate. Asterix indicate a significant difference from control condition: *P<0.05, ** P<0.01, *** P<0.005.

FIG. 6 shows the results of the in vitro hemolysis assay according to example 4. FIG. 6A shows the percentage of hemolysis as a function of concentration of displacer substance sodium hexanoate. FIG. 6B shows the percentage of hemolysis as a function of concentration of displacer substance sodium octanoate. FIG. 6C shows the percentage of hemolysis as a function of concentration of displacer substance sodium decanoate. FIG. 6D shows the percentage of hemolysis as a function of concentration of displacer substance sodium dodecanoate. Asterix indicate a significant difference from control condition: *P<0.05, ** P<0.01, *** P<0.005.

FIG. 7 shows the removal of uremic toxins by the displacer substances according to the invention, as determined according to example 5. FIG. 7A shows the concentration, expressed in μmol/L, of uremic toxin indoxyl sulfate (IS) as a function of perfusion time, expressed in minutes, of sodium octanoate displacer substance. FIG. 7B shows the concentration, expressed in μmol/L, of uremic toxin p-cresyl sulfate (p-CS) as a function of perfusion time, expressed in minutes, of sodium octanoate displacer substance.

FIG. 8 shows the. dialytic removal of IS and p-CS during a simulated seance of hemodialysis according to example 5. FIG. 8A shows the fractional removal of the uremic toxins p-CS and IS. FIG. 8B shows the quantity of uremic toxins p-CS and IS removed from the blood, expressed in μmol/min.

FIG. 9 shows the hematocrit (FIGS. 9A-B), hemolysis (FIGS. 9C-D) and plasma protein levels (FIGS. 9E-F) measured before and after the simulated hemodialysis session according to example 5.

FIG. 10 shows the plasma octanoate concentration in nephrectomized pig perfused with octanoate or vehicle (saline). FIG. 10A represents a graph showing the evolution of octanoate plasma concentration, expressed in μmol/L (y-axis), as a function of time, expressed in minutes (x-axis), before, during and after hemodialysis according to example 6. FIG. 10B represents a graph showing the mean plasma concentration of octanoate, expressed in μmol/L during hemodialysis according to example 6.

FIG. 11 shows the concentration of indoxyl-sulfate (IS) and p-Cresyl-sulfate (p-CS) in effluent dialysate in pig perfused with octanoate or vehicle (saline) according to example 6. FIG. 11A shows the concentration, expressed in μmol/L, of uremic toxin p-cresyl sulfate (p-CS) as a function of hemodialysis time, expressed in minutes (hemodialysis being performed during t=0 min to t=120 min), of octanoate displacer substance. FIG. 11C shows the concentration, expressed in μmol/L, of uremic toxin indoxyl sulfate (IS) as a function of perfusion time, expressed in minutes, of octanoate displacer substance. FIG. 11B shows the mean concentration of p-CS in the dialysate, expressed in μM, using octanoate displacer substance, or only vehicle (saline). FIG. 11D shows the mean concentration of IS in the dialysate, expressed in μM, using octanoate displacer substance, or only vehicle (saline).

FIG. 12 represents the dialytic clearance of indoxyl-sulfate (IS) (FIG. 12A) and p-Cresyl-sulfate (p-CS) (FIG. 12B) in pig perfused with octanoate or vehicle (saline), according to example 6.

### DESCRIPTION OF THE EMBODIMENTS

The present invention relates to a method for removing uremic toxins bound to a protein from the blood of a patient in need thereof, the method comprising the steps of:
a) Introducing a displacer substance in the blood, under conditions in which the displacer substance replaces the uremic toxins bound to the protein, thereby releasing unbound uremic toxins in the blood,
b) Removing the unbound uremic toxin from the blood by dialysis,
   the displacer substance being chosen from (C6-C12)-fatty acids, salts of (C6-C12)-fatty acids, a precursor of said (C6-C12)-fatty acids, or mixtures thereof,
   the displacer substance being provided in the blood at a plasma concentration comprised in the range of from 1 μM to 3 mM, in particular of from 0.5 to 3 mM.

In particular, the patient in need thereof is a patient suffering from kidney disease, in particular acute or chronic kidney disease, more in particular end-stage renal disease or uremic syndrome.

In other words, the present invention relates to (C6-C12)-fatty acids, salts of (C6-C12)-fatty acids, a precursor of said (C6-C12)-fatty acids, or mixtures thereof for use as a displacer substance for removing uremic toxins bound to a protein from the blood of a patient, wherein the displacer substance used is provided in the blood at a plasma concentration comprised in the range of 1 μM to 3 mM, in particular of 0.5 to 3 mM.

In particular, the invention relates to a compound for use as described above, for the treatment of kidney disease, in particular acute or chronic kidney disease, more in particular of end-stage renal disease or uremic syndrome.

As used herein, the expression “conditions in which the displacer substance replaces the uremic toxins bound to the protein” refers to conditions in which the displacer substance is brought into contact with the protein to which the uremic toxins are bound. In particular, the displacer substance may be perfused into the blood of the patient.

The protein to which the uremic toxins are bound may be any plasma protein, in particular albumin, more in particular human serum albumin.

Step b) of removing the unbound uremic toxins from the blood is performed by dialysis techniques, in particular, the dialysis techniques may be chosen from hemodialysis, peritoneal dialysis, hemofiltration or hemodiafiltration.

Step b) is in particular performed by hemodialysis.

Steps a) and b) may be implemented concomitantly, whereby the displacer substance is preferably continuously provided during the process of dialysis.

### Displacer Substance

Uremic toxins may be present in the blood of a patient in the form of a complex with proteins present in the blood, in equilibrium with free, unbound uremic toxin.

By the term “displacer substance” is meant herein a compound able to bind to a protein in the blood, in particular albumin, so as to displace the uremic toxins bound to said protein. In other words, the displacer substance may shift the equilibrium “free uremic toxins-protein bound uremic toxins” such as to result in accumulation of free uremic toxin, and less protein bound uremic toxin. In particular, the displacer substance may be able to displace uremic toxins from the Sudlow's sites I or II on human serum albumin, more in particular from the Sudlow site II of human serum albumin.

The binding affinity of the displacer substance for the protein may be higher than the binding affinity of the uremic toxins for the protein. In particular, the binding affinity for the displacer substance

Alternatively, the binding affinity of the displacer substance for the protein may be lower than the binding affinity of the uremic toxins for the protein, such as 10% of the binding affinity of the uremic toxin.

The lower the binding affinity of the displacer substance for the protein, the higher the concentration of displacer substance that should be used may be.

The binding affinity of the displacer substances and/or the uremic toxins may be determined by molecular modelling. In particular, the binding affinity for the Sudlow's site II on human serum albumin may be determined. Molecular modeling may be implemented using Arguslab (http://www.arguslab.com/arguslab.com/ArgusLab.html) software and PRODIGY webserver (https://bianca.science.uu.nl/prodigy/).

The displacer substances used in the method according to the invention are medium chain fatty acids, chosen from (C6-C12)-fatty acids, salts of (C6-C12)-fatty acids, a precursor of said (C6-C12)-fatty acids, or mixtures thereof.

These substances have unexpectedly been identified by the inventors as having sufficient affinity for proteins in the blood, so as to effectively displace uremic toxins at low plasma concentrations, in particular from albumin. Shorter fatty acids, such as butanoic acid may not have sufficient affinity for the protein to effectively displace the uremic toxins. Longer fatty acids may cause problems related to toxicity, such as hemolysis, i.e. destruction of red blood cells or dyslipidemia.

With «(C6-C12)-fatty acids» is meant fatty acids having a carboxylic acid group attached to an alkyl chain. (C6-C12)-fatty acids are comprised of 6 to 12 carbon atoms, one of which is comprised in the carboxylic acid group. The remaining carbon atoms constitute the alkyl chain. The (C6-C12)-fatty acids can be linear or branched. With (C6-C12)-fatty acids is also meant (C6-C8)-fatty acids, (C6-C10)-fatty acids, (C8-C10)-fatty acids or (C8-C12)-fatty acids.

Linear (C6-C12)-fatty acids are fatty acids in which the alkyl chain to which the carboxylic acid group is attached is linear, the carboxylic acid group being attached to one of the two —CH₃ end-groups of said alkyl chain, said carboxylic acid being a primary carboxylic acid. Thus, with a linear (C6-C12)-fatty acid is meant a compound chosen from hexanoic acid (caproic acid), heptanoic acid (enanthic acid), octanoic acid (caprylic acid), nonanoic acid (pelargonic acid), decanoic acid (capric acid), undecanoic acid (undecylic acid) and dodecanoic acid (lauric acid).

With branched (C6-C12)-fatty acids are meant linear fatty acids as defined above, in which the alkyl chain is further substituted by one or more alkyl groups as defined above. Alternatively, a branched (C6-C12)-fatty acid may also denote a fatty acid in which the carboxylic acid is attached to one of the —CH₂— in the alkyl chain, said carboxylic acid being a secondary carboxylic acid. Examples of branched (C6-C12)-fatty acids include isobutyric acid or isovaleric acid.

According to the invention, the (C6-C12)-fatty acids can be saturated or unsaturated. With saturated fatty acids is meant a fatty acid not having a carbon-carbon double bond in the alkyl chain. An unsaturated fatty acid is a fatty acid having one or more carbon-carbon double bonds in the alkyl chain.

A “salt of (C6-C12)-fatty acids” refers to a (C6-C12)-fatty acid as defined above, in which the carboxylic acid group is in the form of a salt. Examples of salts of fatty acids are sodium salts, potassium salts, lithium salts or ammonium salts. The salt is in particular a sodium salt.

Within the context of the present invention “a derivative of (C6-C12)-fatty acids” refers to a precursor of fatty acids, which in the bloodstream may be converted into the (C6-C12)-fatty acids as defined herein.

In particular can be mentioned glyceryl esters of fatty acids, such as triglyceryl esters, diglyceryl esters or monoglyceryl esters of fatty acids; more in particular triglyceryl esters of fatty acids, such as for example glyceryl trioctanoate (tricaprylin) or glyceryl tridecanoate (tricapryn).

It can also be mentioned phospholipids and lysophospholipids. These compounds comprise respectively 2 or 1 fatty acids linked to a glycerol moeity by an ester bond. They further comprise a phosphate group linked to the glycerol moiety on one of the alcohol groups of the glycerol moiety, through an O—P bond. In case of a lysophospholipid, one of the hydroxyl groups of the glycerol moiety is free, i.e. not esterified or attached to a phosphate group.

Among the phospholipids particularly useful in the context of the present invention can be cited, by way of example, 1,2-Dioctanoyl-sn-glycero-3-phosphocholine or 1,2-Didecanoyl-sn-glycero-3-phosphocholine.

Among the lysophospholipids particularly useful in the context of the present invention can be cited, by way of example, 1-Octanoyl-sn-glycero-3-phosphocholine or 2-Octanoyl-sn-glycero-3-phosphocholine.

The use of such a precursor of (C6-C12)-fatty acids is advantageous because it allows a progressive release of (C6-C12)-fatty acids during step a), during a dialysis session, the derivatives being progressively hydrolysed in the bloodflow especially in the presence of heparine (used as anticoagulant in the bloodlines) that exacerbate the activity of lipoprotein lipase, the enzyme that catalyses the hydrolysis of, for instance, triglycerides into fatty acids. These precursors, used in emulsions, are furthermore demonstrated to be safe for use in human and conforms to regulatory requirements. The precursors of fatty acids may also be used as pre-dialytic bolus instead of continuous perdialytic perfusion.

The displacer substance according to the invention is in particular an unsaturated linear fatty acid as described above, or a salt thereof as described above.

According to an embodiment of the invention, the displacer substance is chosen from hexanoic acid, octanoic acid, decanoic acid and dodecanoic acid or mixtures thereof, in particular octanoic acid or decanoic acid.

According to an embodiment of the invention, the displacer substance is a salt of (C6-C12)-fatty acids, more in particular a sodium salt.

In particular, the displacer substance is sodium octanoate, sodium decanoate or mixtures thereof, more in particular sodium octanoate.

According to an embodiment of the invention, the displacer substance is a precursor of (C6-C12)-fatty acids, wherein said precursor of (C6-C12)-fatty acids is chosen from glyceryl esters of (C6-C12)-fatty acids, phospholipids of (C6-C12)-fatty acids or lysophospholipids of (C6-C12)-fatty acids, in particular triglyceryl esters of (C6-C12)-fatty acids.

In particular, said glyceryl esters are in a composition comprising triglycerides of octanoic acid and decanoic acid, in particular wherein the glyceryl esters comprise about 54 mol % of octanoic acid and about 40 mol % of decanoic acid relative to the total amount of fatty acids of which the esters are comprised, more in particular wherein the composition is in the form of an oil in water emulsion comprising 10% by weight of said glyceryl esters and 10% by weight of soy oil.

A compound useful in this embodiment is the commercial mixture “Medialipid® 20%” (B. Braun Medical), a formulation of medium chain triglycerides and refined soja oil. This commercial mixture comprises mainly triglyceride esters of octanoic acid (54%), decanoic acid (40%), and small quantities of other acids such as hexanoic acid (2%) and dodecanoic acid (4%).

According to a particular embodiment, the displacer substance as described herein may be the only displacer substance used in the method according to the invention, i.e; provided in step a). In other words, the displacer substance may not be used in combination with other displacer substances, such as in particular salicylic acid.

According to this embodiment, the displacer substance may displace the uremic toxins from the protein sufficiently efficiently so as to not need to be combined with other displacer substances.

### Uremic Toxins

Any uremic toxin which binds to plasma proteins, in particular albumin, may be displaced by the displacer substances used in the method of the invention. Examples of such uremic toxins have been disclosed by Duranton et al. (J Am Soc Nephrol. 2012, 23(7), P. 1258-1270).

In an embodiment, the uremic toxins include p-cresyl sulfate (p-CS), p-cresyl-glucuronide (p-CG), indoxyl sulfate (IS), 3-carboxy-4-methyl-5-propyl-2-furanepropionate (CMPF), Hippuric acid, Indole-3-acetic acid, phenyl acetic acid or mixtures thereof.

The displacer substance according to the invention are particularly useful in displacing indoxyl sulfate and p-cresyl sulfate uremic toxins, in particular from albumin.

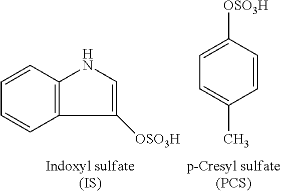

In the invention, the displacer substance is provided in the blood such as to achieve a plasma concentration in the range of from 1 μM to 3 mM, in particular of 0.5 to 3 mM. Within the meaning of the invention, this concentration relates to an initial plasma concentration of the (C6-C12)-fatty acids or salts thereof. Indeed, the concentration of displacer substance may decrease in time due to metabolism of the displacer substances in the blood or due to the dialytic clearance.

In addition, this concentration relates to the amount of fatty acid equivalents administered. Thus in case a precursor is used, the plasma concentration relates to the amount of (C6-C12)-fatty acids susceptible to being released from said precursor. Thus, a triglyceryl ester of (C6-C12)-fatty acids provided at a plasma concentration of 1 mM are considered to correspond to a concentration of about 3 mM of (C6-C12)-fatty acids.

When a mixture of displacer substances is used, the plasma concentration relates to the sum of the concentrations of each individual displacer substance.

In a particular embodiment, the displacer substance is provided in the blood such as to achieve a plasma concentration in the range of 0.5 to 2 mM, more in particular in the range of 0.5 to 1 mM.

In an embodiment, the displacer substance is provided in the blood such as to achieve a plasma concentration in the range of 1 μM to 2 mM, in particular in the range of 1 μM to 1.5 mM, or in the range of 1 μM to 1 mM, or in the range of 1 μM to 0.5 mM.

In an embodiment, the displacer substance may be provided in the blood such as to achieve a plasma concentration in the range of 2 μM to 3 mM, such as 2 μM to 2 mM, 1 μM to 1.5 mM, 2 μM to 1 mM or 2 μM to 0.5 mM or 1 μM to 3 μM.

When using a too low concentration of displacer substance, the displacer substance may not be present in sufficient concentration to effectively displace the uremic toxins from the protein, in particular if the binding affinity of the displacer substance for the protein is lower than the binding affinity of the uremic toxins for said protein.

At too high concentrations, adverse reactions may occur when the displacer substance is provided in the blood of a patient, such as hemolysis.

Thus, according to an embodiment, a triglyceryl ester of (C6-C12)-fatty acid may be provided to the patients blood at a concentration comprised in the range of about 0.33 M to about 1 mM, in particular 0.167 mM to about 1 mM, in particular in the range of about 0.167 to about 0.67 mM, more in particular in the range of about 0.33 to about 0.5 mM, or 0.33 μM to 1 μM.

According to an embodiment, a phospholipid of (C6-C12)-fatty acid may be provided to the patients blood at a concentration comprised in the range of about 0.5 μM to about 1.5 mM, in particular of about 0.25 mM to about 1.5 mM, in particular in the range of about 0.25 to about 1 mM, more in particular in the range of about 0.25 to about 0.5 mM, or 0.5 μM to 1.5 μM.

According to an embodiment, a lysophospholipid of (C6-C12)-fatty acid may be provided to the patients blood at a concentration comprised in the range of about 1 μM to about 3 mM, in particular of about 0.5 mM to about 3 mM, in particular in the range of about 0.5 to about 2 mM, more in particular in the range of about 0.5 to about 1 mM, or 1 μM to 3 μM.

In a particular embodiment, the displacer compound is sodium octanoate, provided at a plasma concentration in the range of 1 μM to 3 mM, in particular in the range of 0.5 to 3 mM.

In a particular embodiment, the displacer compound is sodium decanoate, provided at a plasma concentration in the range of 1 μM to 2 mM, in particular in the range of 1 to 2 mM.

In an embodiment, the displacer substance is a mixture of dodecanoic acid or a salt thereof and one or more (C6-C10)-fatty acids or salts thereof, in particular, wherein the dodecanoic acid or a salt thereof is provided at a plasma concentration of 0.25 mM or less, and the one or more (C6-C10)-fatty acids or salts thereof are provided at a concentration in the range of 0.25 to 2.75 mM, in particular in the range of 0.5 to 2.75 mM.

The displacer substance may be provided to the blood of the patient in the form of a solution or an emulsion, in particular a solution.

The displacer substance may be provided as an aqueous solution of said displacer substance. In particular, in this embodiment, the displacer substance may be chosen from (C6-C12)-fatty acids or salts thereof, as defined above.

Alternatively, the displacer substance may be provided in the form of an emulsion comprising a vegetal oil, such as soy oil, water and optionally an emulsifier such as soy lecithin. In particular in this embodiment, the displacer substance may be a precursor of (C6-C12)-fatty acids, such as a glyceryl derivative as described above.

In a particular embodiment, the aqueous solution in which the displacer substance is provided is an aqueous buffer solution, in particular phosphate-buffered saline (PBS), or an isotonic sodium chloride solution (0.9% w/V).

The aqueous solution in which the displacer substance is provided advantageously has a pH compatible with physiological conditions, i.e. comprised in the range of 7 to 7.5, in particular in the range of 7.3-7.4.

In a particular embodiment, the displacer substance is present in the aqueous solution, or in the emulsion at a concentration of 75 mM to 400 mM, in particular of 75 mM to 350 mM, more in particular of 75 mM to 300 mM.

With “a concentration of 75 mM to 300 mM” should also be understood the following ranges: of 100 mM to 300 mM, of 200 mM to 300 mM, of 75 mM to 200 mM, of 150 mM to 200 mM. In particular, the aqueous solution of displacer substance is a saturated aqueous solution of displacer substance.

The plasma concentration of displacer substance may be achieved by controlling its perfusion flow rate during dialysis and by controlling the amount of displacer substance that is provided to the blood, adjusting the concentration of the displacer substance in the solution which is provided to the patient, and the flow rate into the blood of said solution.

The solution or emulsion comprising the displacer substance may be provided to the blood of the patient at a flow rate of 75 μL/min to 2.5 mL/min, in particular of 75 μL/min to 450 μL/min.

A dialysate may be provided to the dialyzer at a flow rate of 0 ml/min to 1000 ml/min. A flow rate of 0 ml/min may be used in hemofiltration. In hemodialysis or hemodiafiltration, flow rates such as 10 ml/min to 1000 ml/min, in particular 100 ml/min to 1000 ml/min, more in particular 100 ml/min to 500 ml/min may in particular be used.

The dialysate may be any commercially available dialysate, such as bicarbonate or acetate based dialysates containing or not citrate, lactate or chloride.

The blood flow may be provided comprised from 20 ml/min to 500 ml/min.

Dialysis may be performed at a temperature comprised in the range of from 35° C. to 40° C., in particular at the temperature of the human body.

In an embodiment, dialysis is performed for a duration of 1 to 6 hours, in particular from 3 to 5 hours.

The solution or emulsion comprising the displacer substance may be provided extracorporeally. In an embodiment, the displacer substance may be provided in a line, i.e. an “arterial line”, through which blood flows from a patient to a device for removal of the unbound uremic toxins. In an alternative embodiment; the displacer substance may be provided in a line, i.e. a “venous line”, through which blood flows from a device for removal of the unbound uremic toxins to the patients.

The device for removal of the unbound uremic toxins may be a dialyzer in case of hemodialysis, or may be a filter, in the case of hemodiafiltration, or a combination thereof in case of hemodiafiltration.

In other words, the invention relates to a method as described above, wherein the displacer substance is provided extracorporeally, as a solution or an emulsion of said displacer substance, in a line through which blood flows form a patient to a device for removal of the unbound uremic toxins.

In an embodiment, the displacer substance is provided by perfusion.

Alternatively, the solution or emulsion comprising the displacer substance may be provided into the body of the patient, for instance by perfusion. This embodiment may be implemented in case of peritoneal dialysis.

Referring now to FIG. 2, and as an illustrative non-limiting example of a method according to the present invention, a hemodialysis method as described herein above may be carried out using a setup 10 comprising a dialyzer 11 connected to the patient by an arterial line 12 and by a venous line 13. In this setup, blood flows from the patient to the dialyzer 10 through the arterial line 12, and blood flows from the dialyzer 10 back to the patient through the venous line 13. The setup may further comprise a blood flow controller 14 and a dialysate flow controller 15.

A dialysate is provided to the dialyzer 10, and

A displacer substance is provided by perfusion, for instance using a syringe 16 or a peristaltic pump, of a solution, or emulsion; comprising the displacer substance in the arterial line 12.

The dialysate flow-controller 15 allows to control the flowrate of the dialysate to the dialyzer.

The blood flow controller 14 allows to control the flowrate of the blood in the arterial line 12.

The dialysate flow controller (15) allows to control the flowrate of the dialysate.

It has been found that the method according to the invention shows several advantages with respect to prior art methods. Indeed, the displacer substances used in the invention are able to effectively displace uremic toxins from the protein to which they are bound, at low concentrations. The displacer substances used in the invention do not show adverse effects such as extensive hemolysis at the concentrations used and can moreover be easily handled in that they can for instance be administered in the form of a solution or safe biocompatible emulsions.

The following examples serve to illustrate the invention without intending to limit its scope.

### EXAMPLES

### Chemicals

Medium-chain fatty acids (hexanoic, octanoic, decanoic and dodecanoic acids), Human Serum Albumin (reference A9511) and indoxyl-sulfate (reference 13875) were purchased from Sigma-Aldrich (Saint Quentin Fallavier, France). p-Cresyl-sulfate was synthesized according to the method described by Feigenbaum & Neuberg (J Am Chem Soc. 1941. 63 (12): 3529-30). All solvents were purchased from Carlo-Erba reagents (Val-de-Reuil, France) and were HPLC grade.

Competition Assay: Validation of the Displacement of IS with Each Displacer

Competitive interaction of the displacers was confirmed with an ultrafiltration assay under in vitro CKD conditions. In phosphate-buffered saline (pH=7.40), physiological concentration of HSA (500 μM) was incubated for 2 h at room temperature with either indoxyl-sulfate (IS) at maximal achievable concentrations of 250 μmol/L. Then, 1-3 mM of displacer was added to the solution and incubated at 37° C. on a water bath for one hour. Control experiments were performed by adding the same volume of PBS instead of displacer. Then, 600 μL of samples were transferred in filtration devices (Corning®, Spin-X® UF Concentrator, Wiesbaden, Germany, MWCO: 5 kDa) and were centrifuged at 10,000 g for 5 min and 13,000 g for 15 min at room temperature. Ultrafiltrate was collected and free fraction of IS concentration were assayed fluorometrically as described thereafter.

Competition Assay: Validation of the Displacement of p-CS with Each Displacer by Rapid Equilibrium Dialysis (RED)

Rapid equilibrium dialysis is an accurate and reliable method for determining the degree of protein binding. A solution of human serum albumin (500 μM) was spiked with p-CS (250 μM) and incubated for 2 hours at 37° C. The displacer compound was added and sample were further incubated for 1 hour at 37° C. Sample (200 μL) was added to the left chamber of a commercial plate based rapid equilibrium dialysis device (RED, Thermo Fisher, Illkirch, France) while 400 μL of PBS was added to the outer chamber of the RED device. The plate was incubated at room temperature (25° C.) for 6 hours under agitation (400 rpm). The concentration of free p-CS was determined by fluorometric analysis as described below.

### Fluorimetric Assay of Uremic Toxins

Samples were diluted in PBS and fluorescence was measured with a Jasco FP-8300iRM Spectro-fluorometer (Jasco, Tokyo, Japan). The excitation and emission wavelengths were set at 280 nm/383 nm for indoxyl-sulfate and 260 nm/295 nm for p-cresyl sulfate, respectively. Standard curves were run in parallel for indoxyl-sulfate (0-2.5 μmol/L) and p-cresyl-sulfate (0-200 μmol/L).

### In Vitro Hemolysis Assay

One milliliter of fresh bovine blood was incubated for 4 h at 37° C. with 50 μL of a solution of medium chain fatty acids (final concentration: 0.25, 0.5, 1, 2 or 3 mmol/L) or PBS as control. A positive control of hemolysis (taken as 100% hemolysis) was produced by incubating 1 mL of bovine blood with 0.1% (v/v) of TRITON X100 followed by 2 freeze-thaw cycles. Samples were centrifuged 2 min at 9000×g and plasma was stored at −20° C. until free hemoglobin assay. Hemolysis was estimated by the free hemoglobin concentration. Hemoglobin concentration was determined spectrophotometrically as described by Fairbanks et al (Clin Chem. 1992 January; 38 (1): 132-40). Briefly, plasma samples were diluted to 1:10 in Na₂CO₃ 9.4 mmol/L and absorbance was read at 415 nm, 450 nm and 700 nm on a microplate reader (Tecan, Lyon, France).

Free hemoglobin concentration was calculated using the following equation:

$$fHb = 0.01017 \times [ ( 154.7 \times {A}_{415 nm} ) - ( 130.7 \times {A}_{450 nm} ) - ( 123.9 \times {A}_{700 nm} ) ] \times d$$

Wherein:
fHb is the concentration of free hemoglobin in g/L,
A is the absorbance at the specified wavelength,
d is the dilution factor.

Results were expressed as percent of total hemolysis.

### Closed Loop System Using a HD Generator: Hemodialysis of Fresh Bovine Blood

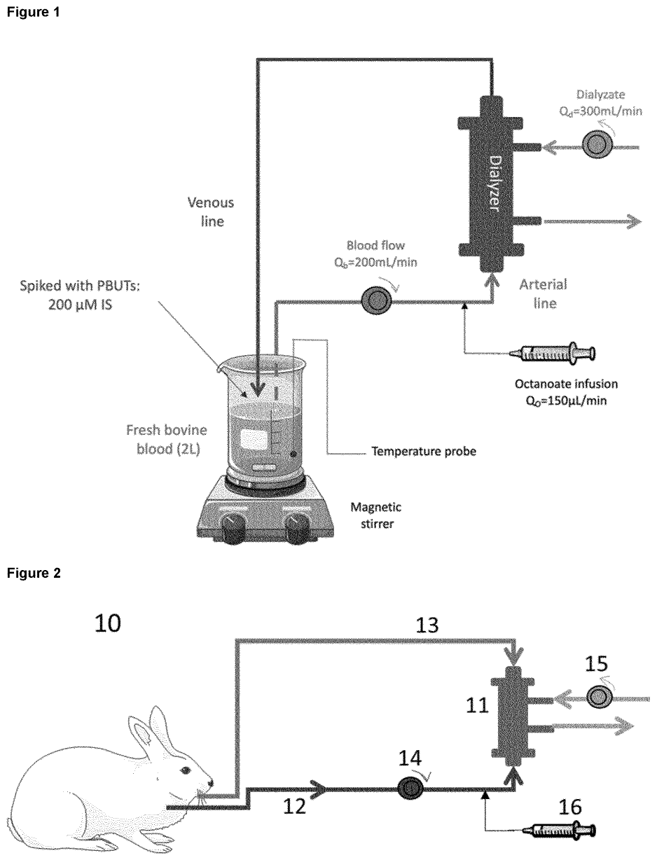

To assess the reality of the displacement and the increase of the removal of PBUT during a hemodialysis session, batches of 20 liters of fresh bovine blood were collected from the slaughterhouse under the authorization VC18731 (Oct. 4, 2018) issued from the Direction Départmentale de la Protection des Populations—Direction des Services Vétérinaires (DDPP-DSV, Lyon, France). Blood was heparinized at 3,750 UI/L with Sodium Heparin (Choay, Cheplapharm, France) and filtered on a nylon mesh. Two liters of blood (2 L) were spiked with 200 μmol/L of IS and gently stirred for 2 hours at 37° C. A 2-hour hemodialysis session was performed with Fresenius 5008 CorDiax hemodialysis generator (Fresenius, Sevres, France) and with a polysulfone-based hemodialysis membrane (FXHDF1000, 2.2 m², Fresenius Medical Care, Bad Homburg, Germany). Arterial and venous port were disposed in the blood batch to perform a closed loop circuit (see representation of the experimental device on FIG. 1). Blood and dialysate flow rates were set at 200 and 300 mL/min, respectively and dialysate temperature was set at 38° C. The infusion of the solution of octanoate (224 mmol/L) as a displacer was made on the arterial line at a flow rate of 150 μL/min (i.e. 33.6 μmol/min). Blood samples were collected from the arterial lines at 0, 15, 30, 45, 60, 75, 90 and 120 minutes. Blood was centrifuged at 9,000 g for 2 minutes and plasma was stored at −20° C. Total concentration of indoxyl-sulfate was assayed by HPLC coupled with fluorescence detection as described below. At the beginning and the end of the experiment, hemolysis was estimated through the measurement of free hemoglobin in plasma as described above. Total protein concentration was measured according to the method of Lowry using bovine serum albumin as standard. Hematocrit was measured using a micromethod (Mondal H, Lotfollahzadeh S. Hematocrit. 2023 Jan. 2. In: StatPearls [Internet]. Treasure Island (FL): StatPearls Publishing; 2023). Briefly, 70 μL of blood was collected in a heparinized capillary tube. The end of the capillary tube was sealed with clay sealant and centrifuged at a rate of 9000 rpm for 5 min. The hematocrit level was read using a microhematocrit card reader.

### HPLC Assay of Protein Bound Uremic Toxins

5-Hydroxyindole-2-carboxylic acid (5-HICA) was used as an internal standard in all experiments. Plasma samples were centrifugated at 17 000 rpm for 5 minutes. 125 μL of the supernatant was then pretreated using the Ostro Pass-through Sample Preparation Device (Waters, Saint-Quentin-en-Yvelines, France), including an in-well protein precipitation with acetonitrile (375 μL) in combination with a pass-through method that allows a good extraction. Eluants were evaporated to dryness in a gentle stream of nitrogen gas. The dry extract was resuspended in 125 μL of mobile phase (composed of 10% (v/v) Methanol, 73.1 mmol/L sodium acetate and 0.3 mmol/L Ethylene diamine tetracetic acid—EDTA-Na₂). After further centrifugation at 25 000 rpm for 10 minutes, 5 μL of the supernatant were injected into an Agilent 1,100 system (Agilent Technologies, Dover, DE, USA) equipped with a C18 column (C18 Synergi 2.5 μm Polar—RP 100A—100×2 mm, Phenomenex, Torrance, CA, USA). Total PBUT concentrations were determined by a reverse phase HPLC with an isocratic flow at 0.2 ml/min. p-CS and IS were determined by fluorescence analysis (excitation λₑₓ: 270 nm; emission λₑₘ: 320 nm). The retention times of 5-HICA, p-CS and IS were 2.3 min, 4.1 min and 6.3 min, respectively. Measurements were calibrated with standard curves for all analytes and were linear over the concentration range of the experiments. The dynamical ranges of calibration were 0.31-40 μmol/L for IS and 1.56-200 μmol/L for p-CS.

### Statistical Analysis

Due to the small number of replicates (n=5-8), all data were compared using non-parametric statistics. Data are presented as median (interquartile range). The statistical significance of the ultrafiltration experiment and the hemolysis measurements were studied with a Kruskall & Wallis followed when appropriated by Dunn's test. The curve of IS and p-CS decrease was built using all data sets and a non-linear model. For each toxin, the comparison of quantity removed and fractional removal were compared using Mann & Whitney U tests. The comparison of Hematocrit, Hemolysis and protein concentration before and after hemodialysis session with control or octanoate (paired data) were compared using a signed rank Wilcoxon test. Differences were considered significant at the p<0.05 level. All statistical analyses were performed using Graphpad Prism (Graphpad software, La Jolla, CA) and open-source R (https://www.r-project.org/) software.

### Molecular Modeling

To estimate equilibrium constant (Kd) for the binding to the Sudlow's site II on human serum albumin, XRD structure from the PDB were obtained for the ligand indoxyl sulfate (PDB code 2BXH), dodecanoate (PDB code 1E7F), decanoate (PDB code 1E7E), and octanoate (PDB code 5X52). For p-Cresyl-sulfate for which no PDB structure is available, docking simulation was performed using Arguslab as software (http://www.arguslab.com/arguslab.com/ArgusLab.html) and the PDB structure of the HSA-indoxyl sulfate (PDB code 2BXH). The docking box was centered on indoxyl sulfate and the docking simulation was achieved with a genetic algorithm with default parameters. For butanoate and hexanoate, their complexes were obtained from the complex of HSA-octanoate (PDB code 5X52) by constructing both ligands butanoate and hexanoate from octanoate leading the corresponding HSA complexes. The ΔG₀ were then estimated using the PRODIGY server by submitting the PDB file of the HSA complexes.

### RESULTS

### Example 1 Molecular Modeling

The binding affinities with respect to Sudlow's site II on human serum albumin was determined using molecular modeling for uremic toxins indoxyl sulfate and p-cresyl sulfate, and of displacer substances hexanoate, octanoate and decanoate. The binding affinities (ΔG₀) were predicted from crystallographic and modeling data using the PRODIGY (PROtein binDIng enerGY prediction) website (https://bianca.science.uu.nl/prodigy/).

Kₐ was computed using the following equation:

$$Δ {G}_{0} = - RT ln {K}_{a} .$$

K_{d} was calculated as 1/Kₐ.

TABLE 1
Estimation of equilibrium constant (Kd) for binding to the Sudlow's site II on human serum
albumin using the corresponding binding mode obtained from XRD or molecular docking.

| | PDB CODE | ΔG₀, | Kₐ, | Kₐ, |
|---|---|---|---|---|
| Compounds | or method | kcal.mol⁻¹ | μM⁻¹ | μM |
| Indoxyl-sulfate | 2BXH | -7.16 | 0.176 | 5.67 |
| p-Cresyl-sulfate | Docking | -7.26 | 0.209 | 4.79 |
| Butanoate | Construction | -6.27 | 0.039 | 25.46 |
| Hexanoate | Construction | -6.87 | 0.108 | 9.25 |
| Octanoate | 5X52 | -7.48 | 0.302 | 3.31 |
| Decanoate | 1E7E | -7.69 | 0.431 | 2.32 |
| Dodecanoate | 1E7F | -8.21 | 1.037 | 0.96 |

Binding affinity (ΔG₀) were predicted from
crystallographic and docking simulations using the
PRODIGY (PROtein binDIng enerGY prediction) website
(https://bianca.science.uu.nl/prodigy/).
Note that the two best predicted ligands are octanoate and decanoate.
Kₐ was calculated using the following equation: ΔG₀ = - RT In Kₐ at 25° C.
Kₐ was calculated as 1/Kₐ.

### Example 2 Displacement of p-CS and IS by Ultrafiltration Assay

The free fraction of IS was measured using ultrafiltration experiments as described as described above. Under baseline conditions, the free fraction of IS was 10.0% (9.3%-14.2%) indicating a major binding to HSA.

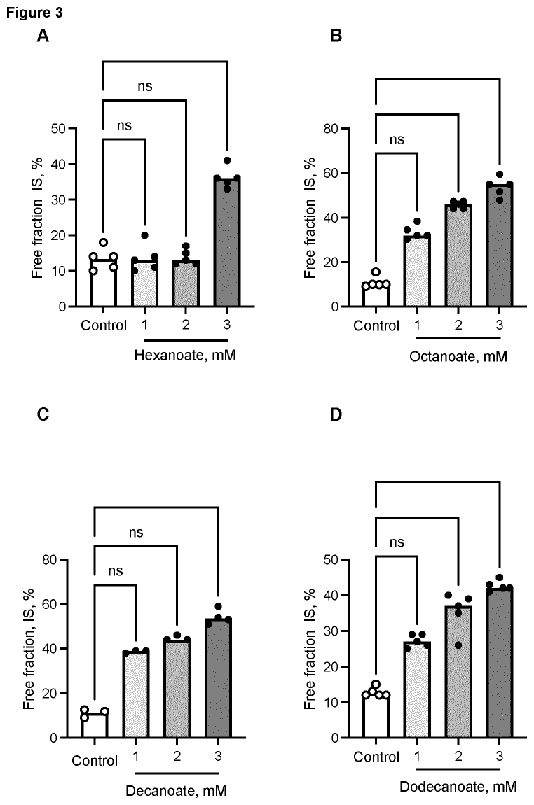

Incubation with hexanoate only increased the free fraction of IS at the highest concentration tested (3 mM) to reach 36% (34%-39%) (P<0.05) (FIG. 3A).

Incubation with octanoate dose dependently increased the free fraction of IS to reach 55% (50%-59%) % (P<0.001), at a concentration of 3 mM (FIG. 3B).

Incubation with decanoate yielded similar results with an increase in the free fraction of IS to reach 54% (52%-58%) at a concentration of 3 mM (P<0.01) (FIG. 3C).

Incubation with dodecanoate dose dependently increased the free fraction of IS 42% (41%-44%) for IS (P<0.001) at a concentration of 3 mM (FIG. 3D).

The free fraction of p-CS was measured using ultrafiltration experiments as described as described above. Under baseline conditions, the free fraction of p-CS was 14% (11%-16%) indicating a major binding to HSA.

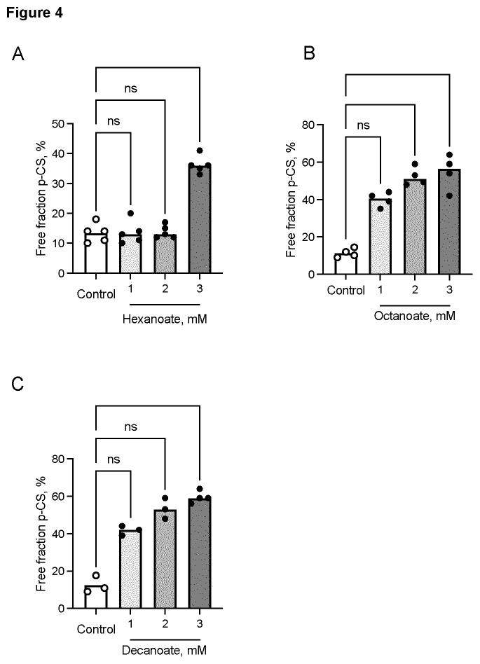

Incubation with hexanoate only increased the free fraction of p-CS at the highest concentration tested (3 mM) to reach 36% (34%-41%) (P<0.05) (FIG. 4A).

Incubation with octanoate dose dependently increased the free fraction of p-CS to reach 57% (45%-63%) % (P<0.001), at a concentration of 3 mM (FIG. 4B).

Incubation with decanoate yielded similar results with an increase in the free fraction of p-CS to reach 59% (57%-63%) at a concentration of 3 mM (P<0.01) (FIG. 4C).

### Example 3 Displacement of p-CS by Rapid Equilibrium Dialysis

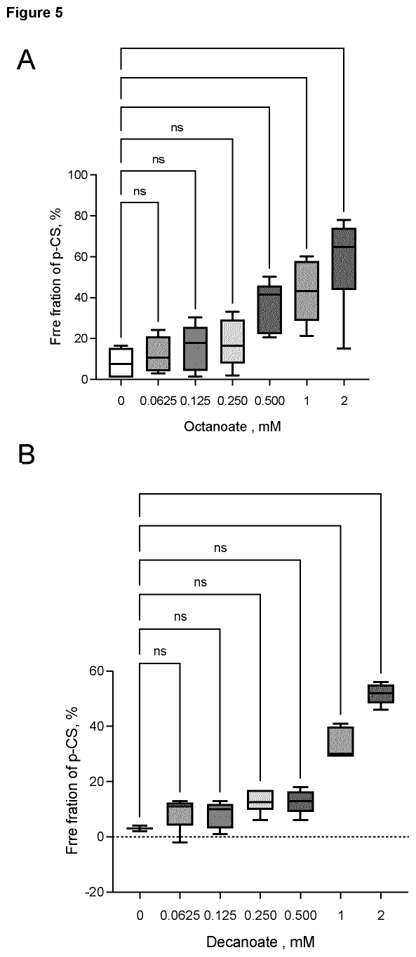

The binding of p-CS (250 μM) to human serumalbumin (500 μM) was measured in the presence of 62.5 μmol/L to 2 mmol/L of octanoate (A) or decanoate (B) at pH 7.4 and 25° C. Free fraction was isolated using rapid equilibrium dialysis (R.E.D devices, Thermo-Scientific) and p-CS was assayed by fluorescence spectroscopy (FIG. 5). Octanoate was shown to effectively displace p-CS when using a concentration of 0.5 mM.

### Example 4 In Vitro Hemolysis Assay

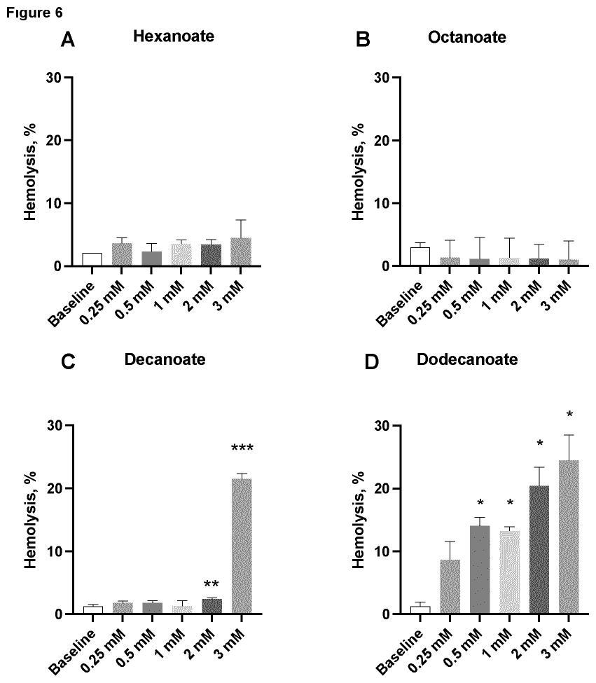

Fresh bovine blood was incubated for 4 h (the full duration of a typical hemodialysis session) at 37° C. in presence of MCFAs (1-3 mmol/L) and dosage of free hemoglobin in plasma was taken as an index of hemolysis (FIG. 6A-D).

Incubation of blood with hexanoic acid (3.6% (2.8%-4.4%), P=0.210) or octanoic acid (2.3% (2.1%-2.5%), P=0.789) did not trigger any significant hemolysis compared to control (1.5% (1.3%-3.0%)). Incubation of bovine blood with decanoic acid triggered hemolysis for a concentration of 3 mmol/L (21.9% (20.9-22.0) %, P<0.001). In contrast, incubation of bovine blood with dodecanoic acid triggered hemolysis for concentrations higher than 0.25 mmol/L (15.3% to 24.9% for 0.5 and 3 mmol/L, P<0.05). Taken together the data indicate that the displacer substances promote hemolysis at an acceptable level for clinical use.

### Example 5 Hemodialysis of Fresh Bovine Blood

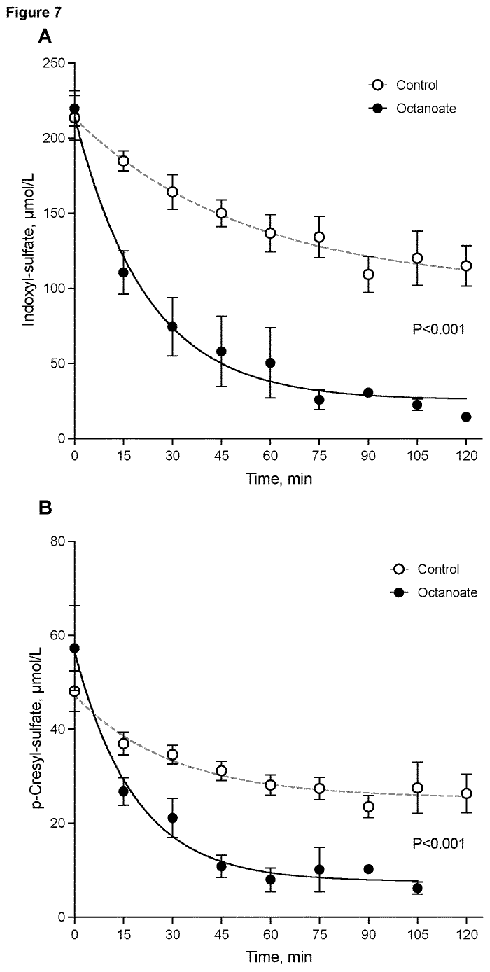

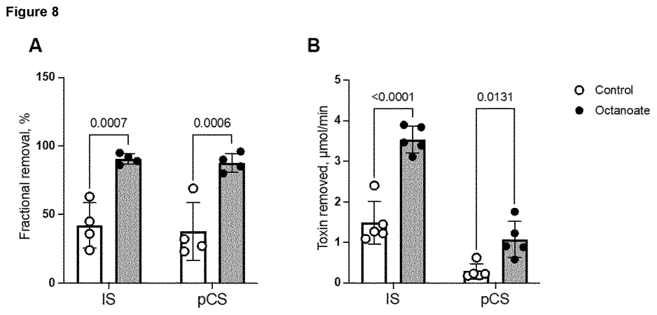

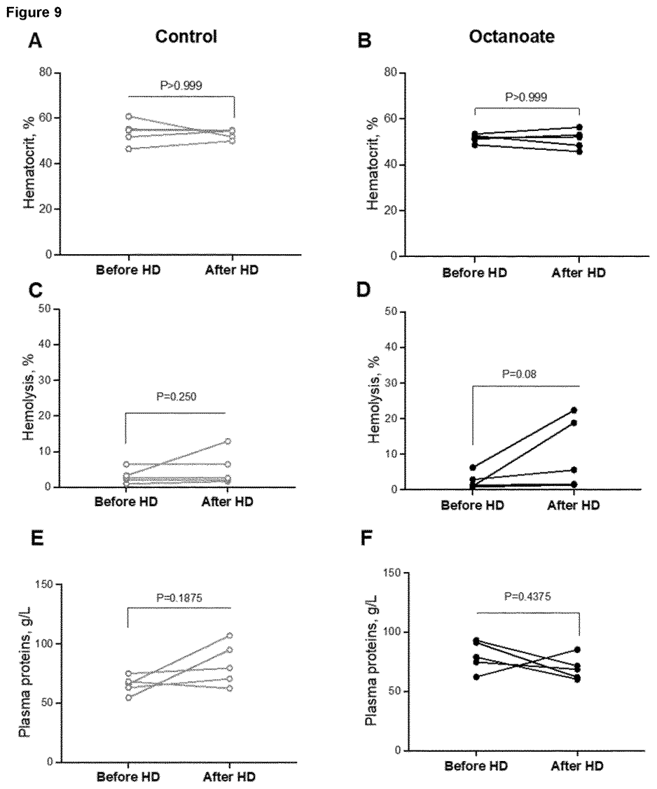

To perform a real-sized proof of concept, bovine blood was spiked with uremic toxins at the concentration observed in patients with ESKD and dialyzed for 2 hours using a clinically used HDF generator (i.e. Fresenius 5008 CorDiax) and HD membrane (FXHDF 1000, Fresenius). A solution of sodium octanoate was continuously infused on the arterial line and compared to saline as a control. The mean temperature of the blood batch (during the whole protocol) was 35.0±0.8° C. and 35.2±1.3° C. for control and octanoate infusion, respectively (U=9, P=0.548). In line perfusion of octanoate strikingly increased the dialytic removal of IS and p-CS as shown in FIG. 7. The p-CS concentration dropped from 48 μmol/L to 30 μmol/L over a 2 h HD session for controls experiments while it decreased from 58 μmol/L to 6 μmol/L in samples treated with octanoate. In good agreement, the IS concentration decreased dropped from 220 μmol/L to 115 μmol/L for controls experiments while it decreased from 214 μmol/L to 14 μmol/L in experiment with continuous octanoate infusion. The half-life of IS in plasma was 36.4 min in the control condition and 15.2 min after octanoate infusion. The half-life of p-CS in plasma was reduced from 20.6 to 12.8 min after octanoate infusion. The toxin removal was increased from 0.3 μmol/min to 1 μmol/min for pCS and 1.5 μmol/min to 3.5 μmol/min for IS with the per-dialytic infusion of octanoate (FIG. 8) The fractional removal of IS and pCS was significantly increased respectively from 36 to 91% (2.5-folds, p<0.001) for IS and from 38 to 88% (2.3-folds, p<0.001) for p-CS. (FIG. 8). The continuous infusion of octanoate for 2 h did not elicit any significant change in hematocrit or hemolysis levels (FIG. 9). These results are good indicators of the absence of major adverse effect of octanoate infusion on bovine red blood cells. No difference was noticed in plasma protein concentration indicating a null net hemofiltration level and excluding that any hemoconcentration occurred.

### Example 6 In Vivo Hemodialysis

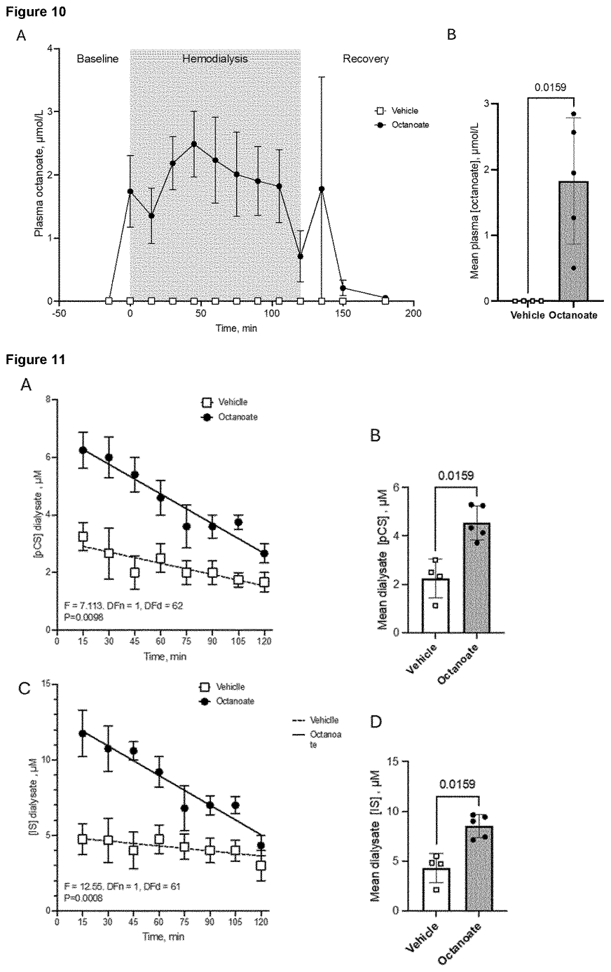

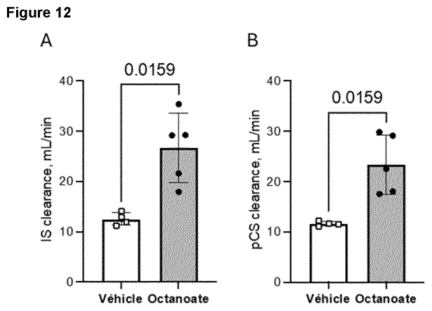

The perfusion of sodium octanoate was tested during an experimental session of hemodialysis in anesthetized pigs. Under sevoflurane anesthesia, pigs (35-40 kg) were catheterized in the 2 jugular veins and one carotid artery. Kidney failure was induced by ligation of both renal pedicles and two protein bound uremic toxins (indoxyl-sulfate—IS and p-cresyl-sulfate—pCS) were injected at 5 mg/kg. Pigs were dialyzed for 2 hours through a 2 lumen jugular catheter using a dialysis generator Physidia S3 with a blood flow of 200 mL/min and a dialysate flow of 200 mL/min. During the HD session, sodium octanoate (333 mmol/L in an aqueous solution of 0.9 w/w % of NaCl) was perfused intravenously at a flow rate of 18 mL/h and perfusion of saline 18 mL/h was used as control. Protein bound uremic toxins were iteratively assayed in plasma and effluent dialysate. Octanoate concentrations were iteratively assayed in plasma and effluent dialysate. Four pigs were perfused with saline (aqueous solution of 0.9 w/w % of NaCl) while 5 pigs were perfused with octanoate. Tolerance of all animals was good and no side effect was noticed (no hemolysis as evidenced by measurement of hematocrit and assay of free hemoglobin). Perfusion of octanoate increased the plasma concentration of octanoate to 2 μmol/L during the whole perfusion (P<0.05, compared to controls, octanoate was assayed using LC-MS/MS) (FIG. 10). Perfusion of octanoate significantly improved the dialytic clearance of both toxins as evidenced by their mean concentration in dialysate (IS+98%, p-CS+103% both P<0.05) (FIG. 11), the fractional and the clearance (IS+113%, p-CS+101%, both P<0.05, assayed in blood collected from the arterial port of the dialyser and effluent dialysate.) (FIG. 12).

In this example, p-Cresyl-sulfate and indoxyl-sulfate were assayed using LC-MS/MS as described in Fabresse et al (Quantification of free and protein bound uremic toxins in human serum by LC-MS/MS: Comparison of rapid equilibrium dialysis and ultrafiltration. Clin. Chim. Acta 2020, 507, 228-235). Briefly, 50 μL of serum sample were spiked with 25 μL of internal standard solution and precipitated with 340 μL of methanol. The solution was homogenized and centrifuged for 10 min at 9000 g, at 4° C. The supernatant was evaporated under nitrogen stream and the dry residue was reconstituted with 80 μL of water. 4 μl were used for injection into the chromatographic system. The LC system consisted of an Ultimate 3000 system (Thermo, Les Ulis, France). Chromatographic separation was performed on an Accucore PFP column (100×2.1 mm, 2.6 μm, Thermo, Les Ulis, France) with a flow rate of 0.5 mL/min at 40° C. The mobile phase consisted of a gradient of 0.1% formic acid in water (A) and pure acetonitrile (B). The gradient was as follow: 1% of B during 1 min, then a gradual increase from 1% of B to 65% in 6.5 min followed by an increase to 90% of B in 0.1 min, stable for a duration of 1.4 min, after which the initial 1% of B is reintroduced allowing an equilibration for a 1.9 min before the next injection. Solutes are detected using a TSQ Quantiva tandem mass spectrometer (Thermo, Les Ulis, France). Ionization is achieved using an electrospray ionization probe alternating between positive (ESI⁺) and negative ionization mode (ESI⁻).Data acquisition and processing were performed using Xcalibur software, version 4.2.28.14 (Thermo, Les Ulis, France).

1. A method for removing uremic toxins bound to a protein from the blood of a patient in need thereof, the method comprising the steps of:
a) Introducing a displacer substance in the blood, under conditions in which the displacer substance replaces the uremic toxins bound to the protein, thereby releasing unbound uremic toxins in the blood,
b) Removing the unbound uremic toxins from the blood by dialysis,
the displacer substance being chosen from (C6-C12)-fatty acids, salts of (C6-C12)-fatty acids, a precursor of said (C6-C12)-fatty acids or mixtures thereof,
the displacer substance being provided in the blood at a plasma concentration comprised in the range of from 1 μM to 3 mM, in particular of from 0.5 to 3 mM.

2. The method according to claim 1, wherein the dialysis is chosen from hemodialysis, peritoneal dialysis, hemofiltration or hemodiafiltration, in particular hemodialysis.

3. (canceled)

4. The method according to claim 1, wherein the displacer substance is a salt of (C6-C12)-fatty acids, in particular a sodium salt.

5. The method according to claim 1, wherein the displacer substance is chosen from hexanoic acid, octanoic acid, decanoic acid and dodecanoic acid or mixtures thereof, or wherein the displacer substance is sodium octanoate, sodium decanoate or mixtures thereof, in particular sodium octanoate.

6. The method according to claim 1, wherein said precursor of (C6-C12)-fatty acids is chosen from glyceryl esters of (C6-C12)-fatty acids, phospholipids or lysophospholipids.

7. The method according to claim 6, wherein said precursor of (C6-C12)-fatty acids is chosen from glyceryl esters of (C6-C12)-fatty acids, said glyceryl esters being in a composition comprising triglycerides of octanoic acid and decanoic acid, in particular wherein the glyceryl esters comprise about 54 mol % of octanoic acid and about 40 mol % of decanoic acid relative to the total amount of fatty acids of which the esters are comprised, more in particular wherein the composition is in the form of an oil in water emulsion comprising 10% by weight of said glyceryl esters and 10% by weight of soy oil.

8. The method according to claim 1, wherein the displacer substance is the only displacer substance introduced in step a).

9. The method according to claim 1, wherein the uremic toxin is chosen from p-cresyl sulfate (p-CS), p-cresyl-glucuronide (p-CG), indoxyl sulfate (IS), 3-carboxy-4-methyl-5-propyl-2-furanepropionate (CMPF), Hippuric acid, Indole-3-acetic acid and phenyl acetic acid, in particular from p-cresyl sulfate or p-indoxyl sulfate.

10. The method according to claim 1, wherein the protein is albumin, in particular human serum albumin.

11. The method according to claim 1, wherein the displacer substance is provided in the blood to achieve a plasma concentration comprised in the range of from 1 μM to 2 mM, in particular in the range of 1 μM to 1 mM, more in particular in the range of 2 μM to 1 mM, or of 1 μM to 3 μM.

12. The method according to claim 1, wherein the displacer substance is provided as an aqueous solution comprising said displacer substance, said displacer substance being in particular chosen from (C6-C12)-fatty acids or salts thereof.

13. The method according to claim 12, wherein the aqueous solution is phosphate-buffered saline or an isotonic solution of sodium chloride.

14. The method according to claim 1, wherein the displacer substance is provided as an emulsion comprising said displacer substance, said displacer substance being in particular a precursor of (C6-C12)-fatty acids.

15. The method according to claim 12, wherein the displacer substance is present in the aqueous solution, or in the emulsion, at a concentration of 75 mM to 400 mM, in particular of 75 mM to 350 mM, more in particular of 75 mM to 300 mM, in particular wherein the aqueous solution is a saturated solution of the displacer substance.

16. The method according to claim 1, wherein the solution or emulsion comprising the displacer substance is provided to the blood at a flow rate of 75 μL/min to 450 μL/min.

17. The method according to claim 1, wherein dialysis is performed at a temperature comprised from 35 to 40° C.

18. The method according to claim 1, wherein the displacer substance is provided extracorporeally, as a solution or an emulsion of said displacer substance, in a line through which blood flows from a patient to a device for removal of the unbound uremic toxins.

19. The method according to claim 18, wherein the device for removal of the unbound uremic toxins is a dialyzer, a filter or a combination thereof.

20. The method according to claim 1, wherein the displacer substance is provided into the blood of the patient by perfusion.

21. The method according to claim 1, wherein the method is carried out with a dialyzer connected to the patient by an arterial line and by a venous line,
wherein blood flows from the patient to the dialyzer through said arterial line, and wherein the blood flows from the dialyzer to the patient through the venous line,
wherein a diazylate is provided to the dialyzer, and
wherein the displacer substance is provided by perfusion in the arterial line.
